(19) United States
(12) Patent Application Publication
(10) Pub. No.: US 2002/0107281 A1
(43) Pub. Date: Aug. 8, 2002

(54) Phototherapeutic and chemotherapeutic immunotherapy against tumors

(75) Inventors: H. Craig Dees, Knoxville, TN (US); Timothy C. Scott, Knoxville, TN (US); Eric A. Wachter, Oak Ridge, TN (US)

Correspondence Address: COOK, ALEX, MCFARRON, MANZO,, CUMMINGS & MEHLER, LTD., Suite 2850, 200 West Adams St., Chicago, IL, 60606, US

(73) Assignee: Photogen, Inc.

(21) Appl. No.: 09/952,448

(22) Filed: Sep. 14, 2001

Related U.S. Application Data
(60) Provisional application No. 60/234,654, filed on Sep. 22, 2000.

(51) Int. Cl. A61K 31/352

(52) U.S. Cl. 514/455

(57) ABSTRACT

The present invention is directed to new methods, medicaments and pharmaceutical compositions and agents for improved cancer treatment that lower recurrence of the primary tumor by causing selective, acute destruction of tumor tissue and thereby exposing the immune system to large amounts of substantially non-denatured tumor material over a short period of time. This effectively vaccinates the host against tumor tissue, allowing the host's immune system to attack any residual tumor tissue, and thereby reduces the recurrence rate and metastasis to remote sites. In preferred embodiments, this enhancement is achieved through application of phototherapeutic or chemotherapeutic modalities that are capable of producing such an acute, necrotic effect on treated lesions without completely denaturing tumor antigens.

### BACKGROUND OF THE INVENTION

The present invention is directed to methods, medicaments and pharmaceutical compositions and agents for cancer and tumor treatment that treat cancers and tumors and enhance the body's immune system to elicit an anti-tumor immune response to these cancers and tumors.

Although evidence is mounting that many, if not all, forms of cancer might be treated by enhancing the ability of the immune system to detect antigenic tumor materials (antigens), unfortunately, current methods of cancer treatment fail to enhance this ability and may in fact significantly suppress immune response to tumor antigens. This negatively affects the ability of the immune system to prevent recurrence of the primary tumor or metastasis to a remote site in the host.

For example, some teachings, such as that taught by Rosenberg and co-workers in U.S. Pat. Nos. 5,126,132 and 5,844,075 wherein tumor infiltrating lymphocytes and related methods are used, claim that melanomas are susceptible to treatment using certain immunologic methods. However, such methods are undesirable because they are based on extracting tumor tissue, processing such tissue to make it antigenic, then reinjecting (inoculating) the resultant antigenic material into the patient. This complex process may fail to destroy large primary or metastatic tumors, and could precipitate adverse side effects.

Because of the fear that exposure to tumor fragments might precipitate spread to remote sites, treatment for many types of tumors normally involves cleanly removing the tumor using a wide margin of excision. While this approach prevents the host from being exposed to tumor materials, it also prevents the immune system from being effectively primed or boosted against the tumor and thereby increases the likelihood of primary tumor recurrence and metastatic progression to a remote site.

In addition, the concurrent use of conventional chemotherapy or radiation modalities, which are frequently used in such cancer treatments, can compound this failure to stimulate an immune response by suppressing systemic immunocompetence.

Traditional cancer treatments also fail to stimulate an immune response because the immune system typically responds proportionally to the intensity and duration of the insult to the host. Thus, a severe, short duration insult is more likely to produce a curative or protective response by the immune system when compared to an insult of lesser intensity that is spread out over a long duration. Slow killing of tumor cells, as is typified by conventional chemotherapy or radiation therapy, fails to afford an acute exposure to antigenic tumor materials. In contrast to the desired acute insult, such mild, protracted exposure can result in desensitization of the immune system to any antigenic materials resulting from such therapy. This phenomenon is similar to that utilized in allergy therapy whereby the host is exposed to low doses of antigen over a long period resulting in enhanced allergen dose tolerance. Thus, these types of cancer treatment modalities may further nullify the ability of any possible anti-tumor immunity to participate in protecting the host against recurrence or metastasis.

It appears that most potential tumor antigens are shielded from exposure to the immune system, preventing a natural anti-tumor response by the immune system. Such shielding is also known to occur with certain infectious agents and is believed to be involved in the formation and persistent development of at least some types of tumors. For example, benign tumors induced by papiloma virus (warts) have antigens that can induce the immune system to destroy the wart. However, these antigens are shielded by some mechanism that prevents the host from recognizing them and destroying the tumor. If these warts are removed cleanly, without causing massive exposure of the shielded antigens to the immune system, recurrence of the wart is likely. However, if warts are removed crudely, whereby the immune system is acutely exposed to the shielded tumor antigens, the rate of recurrence is markedly reduced.

Therefore, current cancer treatment modalities, such as surgical excision with clean and wide margins, or gradual tumor killing using conventional chemotherapy or radiation therapy, fail to adequately expose the immune system to shielded tumor antigens, thereby increasing the likelihood that the tumor will recur.

Prior to the present invention, many advanced, highly selective anti-tumor modalities, such as many photodynamic therapy (PDT) and radiosensitization methods, have been developed that tend to exhibit similar ineffectiveness in anti-tumor immune stimulation, due to their typically mild therapeutic properties. For example, porphyrin-based PDT has proven disappointing in oncology, presumably due to the mild therapeutic mechanism exhibited by photo-activated porphyrins. Similarly, the small enhancements in local radiation toxicity typified by conventional radiosensitizers, such as that based on hyperbaric oxygen or various halogenated radiocontrast materials and nucleic acids, appear to be of insufficient severity to effectively stimulate the immune system. To counter this shortcoming, some investigators, such as for example Nordquist and co-workers (e.g., WO96/31237 and WO99/47162A1) and Korbelik et al. and Chen et al. (e.g., Korbelik, Laser Med. Surg., 14, (1996), 329-334; Korbelik et al., Can. Res, 56, (1996), 5647-565; Korbelik, SPIE, 3914, (2000),16-24; Chen et al., SPIE, 3914, (2000),26-32.), have proposed use of additional immunostimulants as an adjuvant to these therapeutic modalities. These methods have proven largely ineffective and require administration of immune system stimulants having unknown side effects, including possible anaphylactic shock.

Other advanced anti-tumor modalities, such as focused ultrasound, hyperthermia and laser interstitial hyperthermia, laser ablation, and RF or microwave ablation, fail in large measure to provide sufficient selectivity in tumor destruction, and may, due to tissue heating and other physical phenomena, participate in the denaturation of tumor antigens, thereby reducing or destroying any potential for such antigens to stimulate an effective anti-tumor immune response. Further, methods such as laser ablation cleanly remove tumor tissue, precluding immune system exposure to tumor antigens.

Hence, prior methods have failed to achieve adequate acute antigenic exposure for multiple reasons: (1) conventional treatment modalities, such as surgical excision, chemotherapy, and radiation therapy, as currently practiced, fail to expose the immune system to an acute insult from antigenic tumor components; (2) newer treatment modalities, such as PDT and radiosensitization, as currently practiced by others than the present inventors, also fail to expose the immune system to an acute insult from antigenic tumor components; and (3) substantially hyperthermic treatment modalities, such as focused ultrasound, hyperthermia and laser interstitial hyperthermia, laser ablation, and RF or microwave ablation, are capable of precipitating acute tumor destruction but can also substantially denature tumor antigenic materials, thereby rendering such materials ineffective as immune stimulants against viable tumor tissue.

Accordingly, it is an object of the present invention to overcome these failures in prior methods and to develop a simple, highly selective therapeutic modality that is capable of destroying tumors and eliciting an effective anti-tumor immune response, with or without use of additional adjuvants.

### SUMMARY OF THE INVENTION

The present invention is directed to new methods, medicaments and pharmaceutical compositions and agents for improved cancer and tumor treatment that enhances therapeutic response and lowers the recurrence of the primary tumor by causing selective, acute destruction of tumor tissue and thereby enhancing exposure of the immune system to large amounts of substantially non-denatured tumor material over a short period of time. This effectively vaccinates the host against tumor tissue, allowing the host's immune system to attack any residual tumor tissue, and thereby reduces the recurrence rate and metastasis to or at remote sites. These methods, medicaments and pharmaceutical compositions and agents are based on phototherapeutic or chemotherapeutic modalities. Such methods, medicaments and pharmaceutical compositions and agents achieve selective destruction of targeted tumors and effectively enhance immune response to residual or recurrent tumor tissue at the treatment site and at remote sites.

One embodiment of the method of the present invention involves administering a halogenated xanthene or its functional derivative to treat cancers or tumors and to thereby stimulate or enhance the body's immune response to these cancers and tumors.

Another embodiment of the method of the present invention involves administering a halogenated xanthene or its functional derivative to and treating the cancer or tumor using PDT or radiation and thereby stimulating or enhancing the body's immune response to these cancers and tumors.

The present invention is also directed to medicaments and pharmaceutical compositions and agents for treatment of cancers and tumors and for enhancing immune response to cancer or tumor tissues in the body.

The present invention utilizes certain aspects of phototherapy or chemotherapy that are described in further detail in the following co-pending applications: U.S. Ser. No. 09/130,213, filed on Aug. 6, 1998; U.S. Ser. No. 09/096,832, filed on Jun. 12, 1998; U.S. Ser. No. 09/130,041, filed on Aug. 6, 1998; U.S. Ser. No. 09/184,388, filed on Nov. 2, 1998; U.S. Ser. No. 09/216,787, filed on Dec. 21, 1998; U.S. Ser. No. 60/149,015, filed on Aug. 13,1999; U.S. Ser. No. 09/635,276 filed Aug. 9, 2000; U.S. Ser. No. 60/191,803, filed on Mar. 24, 2000; U.S. Ser. No. 60/195,090, filed on Apr. 6, 2000; and U.S. Ser. No. 60/218,464, filed on Jul. 14, 2000, all of which are herein incorporated by reference in their entirety. Specifically, these applications disclose certain therapeutic methods and agents useful for anti-cancer phototherapy or chemotherapy, the expanded uses of which, for the purposes of the present invention, will be made clear by the following example embodiments.

### DETAILED DESCRIPTION OF THE PRESENTLY PREFERRED EMBODIMENTS

The present invention is directed to method and medicaments for enhancement of immune response for treatment of cancers and tumors involving the use of at least one halogenated xanthene. The following embodiments and examples are intended to illustrate but not limit the invention.

Embodiment 1—Enhancement of Immune Response in Phototherapy.

Phototherapy is the treatment of disease by exposure to photons or other energetic emissions. The applied energy may comprise non-penetrating, non-ionizing radiation, such as ultraviolet, visible, or near-infrared light, or penetrating, ionizing radiation, such as x-rays or gamma rays. As a result of the ability of certain pharmaceutical agents to selectively sensitize diseased tissue to the therapeutic effects of applied energy, two related branches of phototherapy have developed:

photodynamic therapy (PDT), which relies upon photochemical reactions resulting from interaction of applied photons with the pharmaceutical; and

radiosensitization, which relies upon pharmaceutical agents capable of mediating transformation of ionizing radiation into locally cytotoxic energy.

These areas, taken together, comprise phototherapy for the purposes of this application.

Certain properties of a new class of phototherapeutic agent, namely the halogenated xanthenes and their functional derivatives, make such agents highly suited for phototherapy (both PDT and radiosensitization), as addressed in the incorporated patent applications. More importantly, the inventors have found that phototherapy of certain tumors using such agents leads to selective acute necrosis of such tumors. This necrosis tends to be substantially more acute and comprehensive in nature than that achieved using conventional phototherapeutic agents and methods, yet appears to leave tumor antigenic material in a substantially non-denatured state. Furthermore, the acute immune system insult or stimulus resulting from such acute necrosis and resultant exposure to such non-denatured tumor antigens stimulates a potent anti-tumoral immune response that effectively vaccinates the host against tumor tissue, and thereby enhances therapeutic response and reduces recurrence rate and metastasis to remote sites.

### EXAMPLE 1

This anti-tumoral effect for the prototypical halogenated xanthene, Rose Bengal, is illustrated by the following example which is not intended to limit the present invention. An adult, female dog with a recurrent malignant fibrous histocytoma (an undifferentiated tumor that could be considered to be a poorly differentiated mesenchymal tumor), approximately 20 cc in volume, was treated by injection of approximately 7 cc of a 10% solution of Rose Bengal at several locations throughout the tumor volume. After a period of five days, an irradiation regimen consisting of 5 daily fractions (2 Gy each) of radiation from a ⁶⁰Co source was begun in order to effect phototherapy of the tumor. Examination of the animal immediately prior to commencement of irradiation indicated a measurable decrease in tumor density along with significant edema and apparent necrosis of large sections of the tumor. Follow-up examination after irradiation (19 days post injection) indicated further measurable decrease in tumor size. Such a response at 5 days is consistent with a chemotherapeutic modality of the injected Rose Bengal, while continued decrease in tumor mass at 19 days is consistent with a phototherapeutic modality related to tumor irradiation. Importantly, additional follow-up examination at 12 weeks post injection indicated significant further reduction in tumor mass. This continued tumor regression is consistent with ongoing immune assault upon remaining tumor tissue. Also notable is the observation at all follow-up intervals of no significant side-effects in healthy tissue surrounding the tumor. Upon final follow-up histologic examination at approximately 12 months post-treatment, no evidence of viable tumor tissue was observed.

In certain tumors, such as melanomas, it is possible to utilize naturally-occurring agents for such phototherapy, as taught in co-pending applications U.S. Ser. No. 09/130,213 and U.S. Ser. No. 09/096,832. Specifically, certain phototherapeutic methods, such as those based on thermal overload, two-photon excitation (TPE) or multi-photon excitation (MPE), are capable of highly selective, acute destruction of melanoma and other pigmented tumors and lesions. Importantly, the inventors have found that such phototherapy of such tumors and lesions using such phototherapeutic methods leads to selective acute necrosis of such tumors and lesions, along with maintenance of non-denatured antigenic components of such treated tumors and lesions. The acute immune system insult and stimulus resulting from such necrosis stimulates a potent anti-tumoral or anti-lesional immune response.

### EXAMPLE 2

This anti-tumoral effect for phototherapeutic treatment of melanoma is illustrated by the following example which is not intended to limit the present invention. Melanoma tumor cell suspensions (10⁴ tumor cells injected subcutaneously into the flanks of C57BL/6 mice) resulted in formation of primary melanoma tumors, within two weeks, at the injection site having a tumor volume of approximately 0.1-1 cm³. The rate of tumor development arising from such implantation was approximately 90% (37/42 mice developed tumors within two weeks of injection). The tumors were treated using MPE photoactivation (using a 500 mW rastered beam of 1047 nm laser light, 1000 J/cm², 120 MHz pulse repetition frequency at approximately 200 fs pulse width). Such photoactivation resulted in selective acute necrosis of treated lesions. Eight mice exhibiting complete response at two weeks after MPE treatment were subsequently challenged using a second injection of melanoma tumor cells (consisting of a single injection of 10³ or 10⁴ tumor cells into the opposite flank). Four weeks following this challenge, 0% (0/8 mice injected with the second dose of melanoma tumor cells) exhibited tumor development. Such dramatic immunity to tumor growth is consistent with a potent immune response stimulated by the acute immune system insult and stimulus from MPE phototherapy of the initial lesion.

### EXAMPLE 3

The immunologic basis for phototherapy-induced immunity is illustrated by the following example which is not intended to limit the present invention. Immunomodulation studies were conducted using serum and tissue samples from melanoma tumor bearing C57BL/6 mice. Serum and tissue samples were collected prior to and following MPE treatment; serum and tissue from untreated, tumor bearing mice was used as a control. Commercial ELISA test kits were used to measure response of specific immunomodulators to MPE phototherapy, such as that described in Example 2. Three modulators were examined: IL-2 (an immune system up-regulator); interferon; and IL-10 (an immune system down-regulator). These data are summarized in Tables 1 and 2. Serum immunomodulator levels were found to remain constant (within the limits of detection of the ELISA tests) for all test conditions. In contrast, intratumoral levels were markedly affected at 24-72 hours post treatment: interferon and IL-2 levels were elevated at 24 hours, and IL-10 was elevated by 72 hours. These findings confirm that the phototherapy modality produces a significant immune response, selectively directed upon tumor tissue, corroborating the observed immunity of such treated animals upon challenge with tumor cells.

Table 1. Comparison of serum immunomodulators levels (measured using standard commercial ELISA tests) for untreated and MPE treated mice having implanted melanoma tumors. No statistically significant difference in serum immunomodulator expression (assessed via two-way ANOVA) is detectable within treatment groups or between treatment groups.

| | Phototherapy | Serum Level (pg/mL) | | |
|---|---|---|---|---|
| Immunomodulator | Treatment | 0 h | 24 h | 72 h |
| Interferon | Untreated | 407 ± 11 | 416 ± 18 | 414 ± 11 |
| | MPE Treated | 417 ± 15 | 415 ± 18 | 416 ± 8 |
| IL-2 | Untreated | 221 ± 17 | 210 ± 26 | 238 ± 36 |
| | MPE Treated | 217 ± 75 | 252 ± 18 | 231 ± 27 |

Table 2. Comparison of intra-tumoral immunomodulators levels (measured using standard commercial ELISA tests) for untreated and MPE treated mice having implanted melanoma tumors. Statistically significant differences in intra-tumoral immunomodulator expression (assessed via two-way ANOVA) are detectable for the MPE treated groups (at 24 and 72 h for interferon and IL-2 and at 72 h for IL-10).

| | Phototherapy | Serum Level (pg/μg protein) | | |
|---|---|---|---|---|
| Immunomodulator | Treatment | 0 h | 24 h | 72 h |
| Interferon | Untreated | 93 ± 17 | 117 ± 45 | 87 ± 29 |
| | MPE Treated | 93 ± 17 | 290 ± 51 | 320 ± 30 |
| IL-2 | Untreated | 127 ± 19 | 156 ± 24 | 143 ± 47 |
| | MPE Treated | 105 ± 34 | 258 ± 23 | 264 ± 19 |
| IL-10 | Untreated | 53 ± 12 | 47 ± 18 | 57 ± 9 |
| | MPE Treated | 57 ± 16 | 64 ± 16 | 230 ± 41 |

### EXAMPLE 4

The immunologic basis for phototherapy-induced immunity is further illustrated by the following example which is not intended to limit the present invention. As an additional control, immune incompetent mice were implanted with subcutaneous tumors, treated using phototherapy, then challenged with a second dose of tumor cells. Since they are immune incompetent, the inventors believed that such mice would respond to phototherapy but fail to develop immunity to tumor cells. Specifically, renal adenocarcinoma (RAG) tumor cell suspensions (10⁴ tumor cells injected subcutaneously into the flanks of BALB/C athymic nude mice) resulted in formation of primary RAG tumors, within five weeks, at the injection site having a tumor volume of approximately 0.1-1 cm³. The rate of tumor development from such implantation was approximately 50% (14/28 mice developed tumors within five weeks of injection). The tumors were treated using intra-lesionally-injected Rose Bengal and green light photoactivation (1% Rose Bengal administered intra-lesionally at approximately 1 cc/cm³ tumor volume, then illuminated 24 hours post administration using 532 nm laser light at 100 J/cm², 200 mW/cm²). Such phototherapy resulted in the predicted selective acute necrosis of treated lesions. Seven mice exhibiting complete response at two weeks after PDT treatment were subsequently challenged using a second injection of RAG tumor cells (a single injection of 10⁴ tumor cells to the opposite flank). Five weeks following this challenge, approximately 50% (3/7 mice injected with the second dose of RAG tumor cells) exhibited tumor development. Thus, as predicted by the inventors, phototherapy using Rose Bengal resulted in successful treatment of the primary tumor as a consequence of its superior properties as a phototherapeutic agent. However, since these nude mice lack an active immune system, they cannot, and did not, exhibit a successful immune response upon subsequent challenge with tumor cells.

The inventors hypothesize that the initial necrotic effect observed using the phototherapeutic methods, medicaments and agents of the present invention are the consequence of acute localized cell damage within treated tissues that does not precipitate complete denaturing of tumor antigens. Such damage is believed to comprise, for example, disruption of cell membranes or other critical cellular structures. This mechanism of damage is in marked contrast to that of most other therapeutic modalities capable of causing acute damage to treated tissues, such as hyperthermia, that typically result in denaturation of antigenic tissue components. Hence, in contrast to conventional therapeutic modalities, the phototherapeutic methods, medicaments and agents of the present invention are especially suitable for stimulation of a host's anti-tumor or anti-lesional immunity through acute exposure of the host's immune system to intact antigenic tumor material. The inventors are submitting these hypotheses to explain their present understanding of how the mechanism work and to make a complete disclosure. However, these hypotheses are not critical for success of the invention nor should they be found to limit the present invention.

Thus, the inventors have demonstrated methods using various phototherapeutic modalities capable of producing an acute, necrotic effect on treated lesions without completely denaturing tumor antigens, and thereby exposing the host's immune system over a short time duration to a high level of such antigens in order to stimulate a potent anti-tumor immune response in the host. Such immune response can thereby be harnessed, for example by phototherapeutic treatment of at least one primary tumor or other lesion, so as to participate in removal of one or more primary tumor or to prevent or remove one or more metastatic lesions. Further, in contrast to prior methods, no adjuvant immunostimulation is required. However, such adjuvant immunostimulation may be employed with the present invention so as to redouble the effects of the anti-tumor immune response.

Accordingly, one preferred embodiment of the present invention is directed to a method for enhancing a host's anti-tumor or anti-lesional immunity through the application of a phototherapeutic modality that is capable of producing an acute, necrotic effect on treated lesions without completely denaturing tumor antigens present in such treated lesions. In a further embodiment, such modality is selected from a group consisting of phototherapy using one or more halogenated xanthenes or their functional derivatives, and thermal overload, TPE or MPE phototherapy of naturally occurring photosensitizers. In yet a further embodiment, such phototherapy with one or more halogenated xanthene or their functional derivatives includes phototherapy with Rose Bengal or its functional derivative. In still another embodiment, such phototherapy with Rose Bengal or its functional derivative include PDT using non-ionizing radiation or radiosensitization using ionizing radiation. Further, an adjuvant immunostimulant can be employed with such phototherapy so as to redouble the effects of such anti-tumor immune response.

In another preferred embodiment, the present invention is directed to phototherapeutic medicaments and pharmaceutical compositions and agents for treatment of cancers and tumors and for enhancing immune response to cancer or tumor tissues in the body. In a further embodiment, such phototherapeutic medicaments and pharmaceutical compositions and agents are selected from a group consisting of one or more halogenated xanthenes or their functional derivatives. In still a further embodiment, such one or more halogenated xanthenes or their functional derivatives comprise Rose Bengal or its functional derivative.

### EXAMPLE 5

The immunologic basis for phototherapy-induced immunity is further illustrated by the following example which is not intended to limit the present invention. Murine hepatoma tumor cells (10⁶ cells, ATCC CRL-1830 Hepa 1-6) were injected subcutaneously into the left flank of immunocompetent mice (C57BL/6). Concurrent with this implantation, a smaller implantation was performed by injecting 5×10⁵ tumor cells into the right flank of these mice. Tumors presented in both flanks within approximately 5-6 days of implantation. One group of mice were otherwise untreated and served as controls. All mice in this group (4/4) were sacrificed when their tumors continued to exhibit unchecked growth. A second group received illumination of the left-flank tumor with green light (532 nm laser light at 100 J/cm², 200 mW/cm²). All mice in this control group (3/3) were sacrificed when all tumors continued to exhibit unchecked growth. A third group received intra-lesional injection of the left-flank tumor with 50 μL of a 10% Rose Bengal solution (the right-flank tumor was not treated); the injected tumor was then illuminated 24 hours post drug administration using 532 nm laser light. This phototherapeutic regimen resulted in eradication of the left-flank tumor in 5/5 mice, and was accompanied by spontaneous rejection of the untreated right-flank tumor in 5/5 mice. Thus, as predicted by the inventors, phototherapy using Rose Bengal resulted in successful treatment of primary tumors. Such treatment also resulted in a systemic anti-tumor immunologic response that proved capable of eradicating an otherwise untreated second, remotely located tumor.

### EXAMPLE 6

The role of non-denatured tumor antigens in phototherapy-induced immunity is further illustrated by the following example which is not intended to limit the present invention. As an additional control, aliquots containing 10⁶ hepatoma tumor cells (murine hepatoma, ATCC CRL-1830 Hepa 1-6) were killed and thermally denatured by boiling for 10 minutes in a standard laboratory PCR machine. These processed cells were then injected subcutaneously into the left flank of immunocompetent mice (C57BL/6). Seven days after this inoculation, 10⁶ viable hepatoma cells (Hepa 1-6) were injected subcutaneously into the right flank of these mice. Within 14 days, the rate of tumor development in the right flank of these mice was 100% (5/5 mice developed tumors in the right flank). A second complement of mice did not receive inoculation with thermally denatured tumor cells but did receive a similar injection of viable hepatoma cells, resulting in a rate of tumor development of 80% (4/5 mice developing tumors at the injection site). Thus, as predicted by the inventors, attempt at establishing anti-tumor immunity through inoculation with thermally-denatured tumor tissue did not prove effective. Such failure is in stark contrast to results obtained with benefit of the present invention, which illustrates that potent anti-tumor immune system response results from in situ treatment of tumor tissue using the phototherapeutic methods, medicaments and agents of the present invention that do not precipitate complete denaturing of tumor antigens.

Embodiment 2—Enhancement of Immune Response in Chemotherapy

The inventors have found that certain chemotherapeutic agents are capable of producing an acute, localized necrotic effect on treated lesions without completely denaturing tumor antigens. Such chemotherapeutic effects may be used to enhance a host's anti-tumor or anti-lesional immunity, and due to their local effects have minimal deleterious impact on the host's overall immune system competence. Specifically, certain special properties and uses of a new class of chemotherapeutic agent, namely the halogenated xanthenes and their functional derivatives, make such agents well suited for lesion-specific chemotherapy upon direct intra-lesional injection. Such chemotherapeutic effects are taught in co-pending application U.S. Ser. No. 60/218,464. Importantly, the present inventors have found that chemotherapy of certain tumors using such agents leads to selective acute necrosis of such tumors. This necrosis tends to be substantially more acute and comprehensive than that achieved using conventional chemotherapeutic agents and methods, yet appears to leave tumor antigenic material in a substantially non-denatured state. Furthermore, the acute immune system insult and stimulus resulting from such acute necrosis and resultant exposure to such non-denatured tumor antigens stimulates a potent anti-tumoral immune response that effectively vaccinates the host against tumor tissue, thereby enhancing therapeutic response and reducing the recurrence rate and metastasis to remote sites.

### EXAMPLE 7

This chemotherapeutic and immunologic anti-tumoral effect for the prototypical halogenated xanthene, Rose Bengal, is illustrated by the following example which is not intended to limit the present invention. An adult, female dog with multiple superficial mast cell tumors was treated by injection of several cc of a 10% solution of Rose Bengal into each of several tumors (approximately one third of the total tumors were treated in this manner). Over the subsequent three month period, all mast cell tumors, both treated and non-treated, exhibited marked and progressive reduction in size. Such a response in the treated tumors is consistent with the expected chemotherapeutic modality of the injected Rose Bengal, while the noted decrease in non-treated tumors represents an ongoing immune assault upon remaining remote tumor tissues. Also notable is the observation at all follow-up intervals of no significant side-effects in healthy tissue surrounding the treated tumors nor any evidence of significant systemic side effects.

### EXAMPLE 8

The chemotherapeutic and immunologic anti-tumoral effect for the prototypical halogenated xanthene, Rose Bengal, is further illustrated by the following example which is not intended to limit the present invention. Immune competent mice (C57BL/6 mice) and immune incompetent mice (BALB/C athymic nude mice) were implanted with subcutaneous RAG tumors, as previously described in Example 4. Upon formation of primary tumors having a tumor volume of approximately 0.1-1 cm³, these two groups of mice were treated by intra-lesional injection of a 10% solution of Rose Bengal (at approximately 1 cc/cm³ tumor volume). This chemotherapeutic treatment resulted in immediate tumor necrosis in both treatment groups. However, upon follow-up, a 100% cure rate (6/6 mice treated) was noted for the immune competent mice, while the cure rate for immune incompetent mice was 0% (0/4 mice treated). Thus, as predicted by the inventors, chemotherapy using Rose Bengal resulted in successful treatment of tumors in immune competent mice, but was less effective in immune incompetent mice due to recurrence (such mice cannot exhibit a successful immune response to any residual tumor cells remaining after initial treatment and are thus susceptible to recurrence).

The inventors hypothesize that the initial necrotic effect observed using the chemotherapeutic methods, medicaments and agents of the present invention are the consequence of acute localized cell damage within treated tissues that does not precipitate complete denaturing of tumor antigens. Such damage is believed to comprise, for example, disruption of cell membranes or other critical cellular structures. Hence, the chemotherapeutic methods, medicaments and agents of the present invention are especially suitable for stimulation of a host's anti-tumor or anti-lesional immunity through exposure of the host's immune system to intact antigenic tumor material. The inventors are submitting these hypotheses to explain their present understanding of how the mechanisms work and to make a complete disclosure. However, these hypotheses are not critical for success of the invention nor should they be found to limit the present invention.

Thus, the inventors have demonstrated methods using chemotherapeutic modalities capable of producing an acute, localized necrotic effect on treated lesions, without completely denaturing tumor antigens, and thereby exposing the host's immune system over a short time duration to a high level of such antigens, to stimulate a potent anti-tumor immune response in the host. Such immune response can thereby be harnessed, for example by chemotherapeutic treatment of at least one primary tumor or other lesion, so as to participate in removal of the primary tumor or to prevent or remove metastatic or other remote lesions. In contrast with prior art, no adjuvant immunostimulation is required. However, such adjuvant immunostimulation may be employed with the present invention so as to redouble the effects of the anti-tumor immune response.

Therefore, another preferred embodiment of the present invention is directed to a method for enhancing a host's anti-tumor or anti-lesional immunity through the application of a chemotherapeutic modality that is capable of producing an acute, localized necrotic effect on treated lesions without completely denaturing tumor antigens present in such treated lesions. In a further embodiment, such modality comprises chemotherapy with one or more halogenated xanthene or their functional derivatives. In yet a further embodiment, such chemotherapy includes chemotherapy with Rose Bengal or its functional derivative. In still a further embodiment, such chemotherapy with Rose Bengal or its functional derivative includes intra-lesional administration of such chemotherapy into one or more lesions to be treated. Further, an adjuvant immunostimulant can be employed with such chemotherapy so as to redouble the effects of such anti-tumor immune response.

In another preferred embodiment, the present invention is directed to chemotherapeutic medicaments and pharmaceutical compositions and agents for treatment of cancers and tumors and for enhancing immune response to such cancer or tumor tissues in the body. In a further embodiment, such chemotherapeutic medicaments and pharmaceutical compositions and agents are selected from a group consisting of one or more halogenated xanthenes or their functional derivatives. In still a further embodiment, such one or more halogenated xanthenes or their functional derivatives comprise Rose Bengal or its functional derivative.

These examples are provided for illustrative purposes, as the present invention is not limited to the recited examples and includes other therapeutic modalities known to those skilled in the art. For example, it will be clear that combinations of any of the example therapeutic modalities discussed herein with other therapeutic modalities, including conventional chemotherapy, radiation therapy, radio frequency ablation, cryotherapy, hyperthermia, or any other combination of such therapeutic modalities, are within the scope of the presently described invention, wherein such modalities may be used to augment the efficacy of the present invention or wherein the present invention may be used to augment the efficacy of such modalities.

We claim:

1. A method for enhancement of immune response in phototherapy for treatment of cancers and tumors, said method comprising the steps of:
administering a halogenated xanthene agent to a patient;
treating said cancers and tumors with phototherapy to cause selective necrosis of a portion of said cancers and tumors, while leaving tumor antigenic material present in said treated cancer and tumors in a substantially non-denatured state; and
thereby stimulating a potent anti-tumoral immune response by the body to effectively vaccinate the body against said cancers and tumors.

2. The method of claim 1 wherein said phototherapy uses applied ionizing radiation.

3. The method of claim 1 wherein said phototherapy uses applied non-ionizing radiation.

4. The method of claim 1 wherein said halogenated xanthene is Rose Bengal.

5. A method of enhancement of a human or animal's anti-tumor immunity by applying a phototherapeutic modality to produce an acute, necrotic effect on a treated tumor without completely denaturing tumor antigens present in said tumor.

6. The method of claim 5 wherein said phototherapy uses applied ionizing radiation.

7. The method of claim 5 wherein said phototherapy uses applied non-ionizing radiation.

8. The method of claim 5 wherein said method includes administering a halogenated xanthene to said human or animal.

9. The method of claim 8 wherein said halogenated xanthene is Rose Bengal.

10. The method of claim 5 wherein said tumors contain naturally-occurring photosensitive agents.

11. The method of claim 10 wherein said phototherapy utilizes thermal overload of said cancers and tumors to cause said selective necrosis of a portion of said cancers and tumors.

12. A medicament comprising at least one halogenated xanthene as a primary active component, wherein said medicament is useful for enhancing immune response in phototherapeutic treatment of human and animal tissue cancers and tumors.

13. The medicament of claim 12 wherein said halogenated xanthene interacts with applied ionizing radiation.

14. The medicament of claim 12 wherein said halogenated xanthene interacts with applied non-ionizing radiation.

15. The medicament of claim 12 wherein said halogenated xanthene is Rose Bengal.

16. Use of a halogenated xanthene in the preparation of a medicament for enhancement of phototherapeutic treatment of human and animal cancers or tumors.

17. The use of claim 16 wherein said phototherapeutic treatment uses applied ionizing radiation.

18. The use of claim 16 wherein said phototherapeutic treatment uses applied non-ionizing radiation.

19. The use of claim 16 wherein said halogenated xanthene is Rose Bengal.

20. Use of a halogenated xanthene comprising:
administering a therapeutically effective amount of a halogenated xanthene into or proximate to a cancer or tumor for phototherapeutic treatment and enhancement of immune response to said cancer or tumor.

21. The use of claim 20 wherein said phototherapeutic treatment uses applied ionizing radiation.

22. The use of claim 20 wherein said phototherapeutic treatment uses applied non-ionizing radiation.

23. The use of claim 20 wherein said halogenated xanthene is Rose Bengal.

24. A method for enhancement of immune response in chemotherapy for treatment of cancers and tumors, said method comprising the steps of:
administering a halogenated xanthene agent to a patient so as to produce selective necrosis of a portion of said cancers and tumors;
thereby stimulating a potent anti-tumoral immune response by the body to effectively vaccinate the body against said cancers and tumors.

25. The method of claim 24 wherein said halogenated xanthene is Rose Bengal.

26. A method of enhancement of a human or animal's anti-tumor immunity by applying a chemotherapeutic modality to produce an acute, necrotic effect on a treated tumor without completely denaturing tumor antigens present in said tumor.

27. The method of claim 26 wherein said method includes administering a halogenated xanthene to said human or animal.

28. The method of claim 26 wherein said halogenated xanthene is Rose Bengal.

29. A medicament comprising at least one halogenated xanthene as a primary active component, wherein said medicament is useful for enhancing immune response in chemotherapeutic treatment of human and animal tissue cancers and tumors.

30. The medicament of claim 29 wherein said halogenated xanthene is Rose Bengal.

31. Use of a halogenated xanthene in the preparation of a medicament for enhancement of chemotherapeutic treatment of human and animal cancers or tumors.

32. The use of claim 31 wherein said halogenated xanthene is Rose Bengal.

33. Use of a halogenated xanthene comprising:
administering a therapeutically effective amount of a halogenated xanthene into or proximate to a cancer or tumor for chemotherapeutic treatment and enhancement of immune response to said cancer or tumor.

34. The use of claim 33 wherein said halogenated xanthene is Rose Bengal.
